(19) United States
(12) Patent Application Publication
(10) Pub. No.: US 2010/0249846 A1
(43) Pub. Date: Sep. 30, 2010

(54) VARIABLE HEIGHT, MULTI-AXIAL BONE SCREW ASSEMBLY

(76) Inventor: Peter M. Simonson, Longboat Key, FL (US)

Correspondence Address: DUANE MORRIS LLP - (prev. San Francisco);IP DEPARTMENT, 30 SOUTH 17TH STREET, PHILADELPHIA, PA, 19103-4196, US

(21) Appl. No.: 12/731,116

(22) Filed: Mar. 24, 2010

Related U.S. Application Data
(60) Provisional application No. 61/163,313, filed on Mar. 25, 2009.

(51) Int. Cl. A61B 17/70; A61B 17/86; B23P 11/00

(52) U.S. Cl. 606/264; 606/305; 29/428

(57) ABSTRACT

A variable height, multi-axial bone screw assembly including a bone screw, a collet, a tulip shaped connector, a crown member, a rod and a set-screw. The bone screw has threads at its lower end and a generally cylindrical shaft at its upper end. The collet has a cylindrically shaped lower end to hold and grasp the bone screw shaft as well as a generally bulb shaped upper end to be received by the tulip shaped connector. By changing the position of the bone screw shaft grasped by the collet, the height of the bone screw vis-à-vis the tulip shaped connector can be varied. All the elements of the bone screw assembly are combined together by placing the collet head, crown member and rod within a central bore of the connector and firmly tightening them in place using the set screw.

This application claims the benefit of U.S. Provisional Patent Application No. 61/163,313, filed Mar. 25, 2009, which application is incorporated herein by reference in its entirety.

### BACKGROUND OF THE INVENTION

The present invention relates to a bone screw assembly for correcting misaligned spinal vertebrae. In particular, the present invention provides an assembly where the bone screws can not only be set at different axes vis-à-vis their connecting rods, but can also be set at different heights vis-à-vis such rods.

Nerve compression and pain can be caused when vertebrae in the spine become misaligned. Spondylolisthesis, for example, is a condition where vertebrae become misaligned by slipping over one another either forwards (anterolisthesis) or backwards (retrolisthesis). Surgical techniques can be used to correct such misalignments. In one such surgical technique, bone screws are affixed to various vertebrae and, through suitable connectors, commonly attached to one or more rods. Often, two sets of bone screw/rod assemblies are created in this way on either side of the spinous process. In these assemblies, the rods are shaped to the desired orientation or angulation of the spinal vertebrae. For example, the rod can be bent to form a normal kyphotic curvature for the thoracic region, or a lordotic curvature for lumbar region of the spine. Over a period of time, the rods apply pressure to the vertebrae until the vertebrae realign themselves in a proper orientation. This is known as a rod-type spinal fixation system. It is system where the desired angulation of the spinal vertebrae is achieved by the shape of the rod. In other words, a plurality of fixation devices including hooks, clamps, bolts and screws simply attach segments of the spine to a fixed rod that over time and with adjustments, as needed, corrects the spine's angulation.

There have been many attempts to create bone screw assemblies for realigning spinal vertebrae. One bone screw assembly that has been in common commercial use is described in Sherman et al.'s U.S. Pat. No. 5,885,286 (“Sherman patent”), the disclosure of which is hereby incorporated by reference. The Sherman patent discloses a relatively simple bone screw assembly featuring a bone screw, a receiver member, a compression member and a connector rod. The bone screw in the Sherman patent is cast with a spherically shaped head at its top end so that it can pivot along different axes when it is affixed to Sherman's receiver member. Sherman's receiver member also holds a transverse rod that can be fitted into a number of other receiver members. By tightening a compression member, such as a set screw, at the top of the receiver member, one can simultaneously lock both the rod and bone screw in position.

While the Sherman bone screw assembly has been used in many surgeries, it has drawbacks and limitations. One such drawback is illustrated in FIG. 1. Because Sherman's spherically shaped head is formed integrally with the remainder of his bone screw, the height of Sherman's bone screw vis-à-vis Sherman's receiving member cannot be varied. This lack of variability can cause Sherman's receiving member to be twisted as it tries to grip onto the rod as shown in the left and right bone screw assemblies illustrated in FIG. 1. With this twisting, the compression member contacts the rod at an angle which leaves gaps between the compression member and the rod. These gaps can create undesirable looseness in the bone screw assembly.

Another multi-axial bone screw assembly is illustrated in Altarac's U.S. Pat. No. 7,163,538 (“Altarac patent”). The Altarac bone screw assembly consists of a complicated arrangement of a bone screw, a post member, a locking cap, a rod connector, a locking nut and a rod. The Altarac patent allows the bone screw to pivot on multiple axes vis-à-vis the rod connector by forming a cage on top of the bone screw that receives a ball shaped engagement member at one end of the intermediate post member. The other end of the intermediate post member is then attached to the rod connector by a combination of a locking cap and locking nut. While, at first glance, it appears that the height of Altarac's bone screw could be adjusted vis-à-vis the rod connector, FIG. 4 of the Altarac patent appears to show that the bone screw will be locked at a fixed, unvarying height vis-à-vis the rod connector. Moreover, due to its complicated arrangement of parts, the Altarac bone screw assembly would be difficult to use during surgery and, if improperly used, could easily lead to dangerous problems.

The object of the present invention is to provide a variable height, multi-axial bone screw assembly that allows improved angulation of bone screws vis-à-vis a fixed cylindrical rod. Such a bone screw assembly can advantageously be used for correction of abnormal cervical, thoracic and lumber curvatures.

### BRIEF SUMMARY OF THE INVENTION

The present invention provides a simple, variable height multi-axial bone screw assembly that allows bone screws to engage a fixed cylindrical rod in any degree of angular orientation or direction. In a preferred embodiment, the bone screw assembly of the present invention includes a bone screw, a collet, a tulip shaped connector, a crown member, a locking washer, a rod and a set-screw. In this embodiment, the bone screw has a threaded shank at its lower end and a generally cylindrical shaft at its upper end. The collet has a cylindrically shaped lower end and a generally bulb shaped upper end which is designed to be received by the tulip shaped connector. This collet also has an interior bore which allows the collet to slide over the bone screw shaft like a sleeve. By changing the amount of the bone screw shaft covered by the collet, one can vary the height of the bone screw vis-à-vis the tulip shaped connector.

The tulip shaped connector has a central bore that is designed, at its lower end, to receive the collet bulb. Within the tulip shaped connector, the locking washer can be placed below the collet bulb and the crown member can be placed above the collet bulb to reliably secure the collet bulb into the lower portion of the tulip shaped connector bore. Once the locking washer, collet bulb and crown member are assembled in the lower portion of the tulip shaped connector bore, the rod can be transversely placed above the crown member in a U-shaped channel within the tulip shaped connector. A set screw can then be tightened above the rod in the tulip shaped connector to simultaneously lock the rod, crown member, collet, locking washer and bone screw into their desired positions. Before the set screw is tightened, the collet bulb can be turned within the tulip shaped connector bore along multiple axes to achieve a desired axial orientation of the bone screw vis-à-vis the tulip shaped connector. To help the collet fit snugly around the bone screw, longitudinal slots are provided along the surface of the collet allowing the solid sections of the collet bulb to collapse around the bone screw shaft when the set screw of the tulip shaped connector is tightened. As a further aid to preventing the bone screw from slipping out of the collet after the bone screw assembly of the present invention has been implanted, the bone screw can be tapered outward.

### BRIEF DESCRIPTION OF THE DRAWINGS

FIG. 1 shows a cross-section view of three multi-axial bone screw assemblies of the type shown in Sherman's U.S. Pat. No. 5,885,286 attached to a spinal rod;

FIG. 2 shows an exploded view of a preferred bone screw assembly of the present invention;

FIG. 3 shows a cross-section, close-up view of the FIG. 2 bone screw assembly when fully assembled, but not yet tightened with the set screw.

FIG. 4 shows the same cross-section, close-up view of the FIG. 2 bone screw assembly after it has been fully assembled and tightened with the set screw.

FIG. 5 shows three multi-axial bone screw assemblies of the present invention attached to a spinal rod where the bone screws are set at different heights.

FIG. 6 shows a cross-section close-up view of an alternative embodiment of the bone screw assembly of the present invention where the bone screw is tapered at its upper end.

FIG. 7 shows a top elevation view of a preferred tulip shaped connector.

FIG. 8 shows a side elevation view of a preferred tulip shaped connector.

FIG. 9 shows a top elevation view of a preferred crown member.

FIG. 10 shows a side elevation view of a preferred crown member.

FIG. 11 shows a perspective view of a preferred collet.

### DETAILED DESCRIPTION OF THE INVENTION

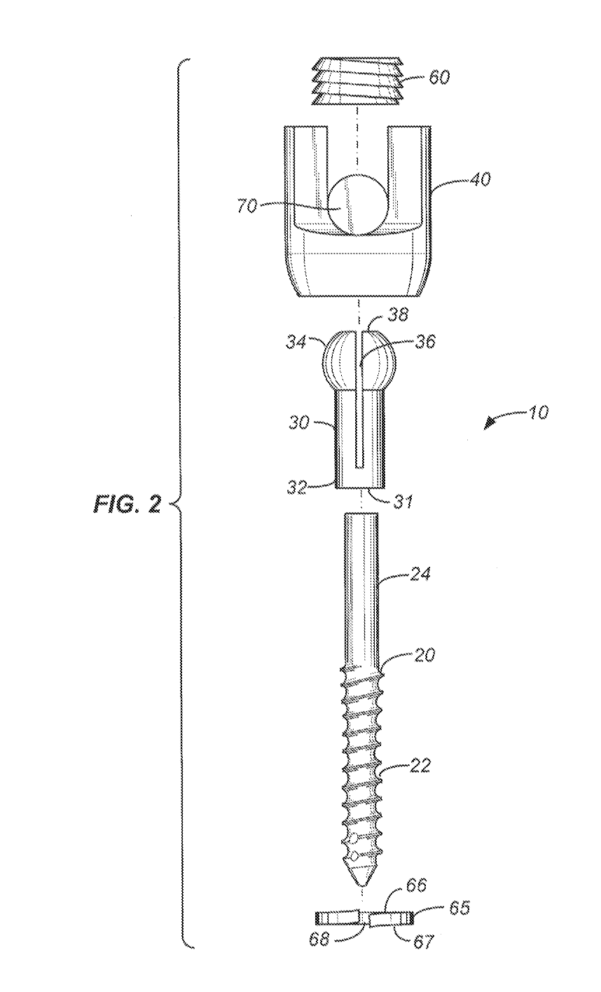

Referring now to FIG. 2, a preferred embodiment of the bone screw assembly 10 of the present invention is shown in exploded form. Components of this bone screw assembly 10 embodiment include a bone screw 20, a collet 30, a tulip shaped connector 40, a crown member 50 (see FIG. 3), an optional locking washer 65, a rod 70 and a set screw 60. The components of the bone screw assembly 10 can be metallic, such as titanium, titanium alloy or stainless steel, or non-metallic, such as PEEK or other types of plastics, or a combination thereof.

The bone screw 20 in this preferred embodiment has a threaded shank 22 at its lower end with threads configured to solidly anchor the bone screw within a bone. Preferably, the threads are cancellous threads, or threads readily adapted for solid fixation within the cancellous bone of the vertebral body. It is understood that the threaded shank 22 can have a variety of configurations depending upon the nature of the bone within which the bone screw 20 is engaged. Moreover, the length of the threaded shank 21 can be adjusted depending upon the bone within which the screw is driven. In one embodiment, the threaded shank 22 has a length of about 1.75 inches, and is configured with threads to engage the pedicle of a lumbar vertebra.

The bone screw 20 further includes a generally cylindrical shaft 24 at its upper end. The diameter of this generally cylindrical bone screw shaft 24 is selected to allow the shaft 24 to fit smoothly within the interior bore 31 of a collet 30 without leaving too much space between the interior surface of the collet 30 and the exterior surface of the bone screw shaft 24. As those of skill in the art will recognize, the bone screw shaft 24 can be formed in other shapes besides cylindrical. For example, the bone screw shaft 24 could also be formed in non-cylindrical shapes such as hexagonal, octagonal or oval cross-sectional shapes. In such case, it would also be advantageous to form the interior bore 31 of the collet 30 in a matching hexagonal, octagonal or oval shape.

Bone screw 20 and collet 30 can be separate pieces (see, FIG. 2) or one-piece (not shown). As separate pieces, the collet 30 and tulip shaped connector 40 may slide over the bone screw 20 (see, FIG. 3). In another separate piece embodiment, the bone screw 20 may possess a head (not shown) to help it attach to collet 30 and tulip shaped connector 40. In a one-piece embodiment, the bone screw 20, collet 30 and tulip shaped connector 40 may be one contiguous piece.

FIG. 6 illustrates a further alternative embodiment for the shape of the upper shaft 82 of the bone screw 80. In this embodiment, the upper shaft 82 is tapered so that the diameter at the top of the shaft 84 is greater than the diameter at the bottom of the shaft 86 (i.e., where it meets the threaded shank 87). For this embodiment, the collet's lower end 92 should be formed with a matching reverse taper so that the upper shaft 82 of the bone screw 80 continues to fit comfortably, but snugly, into the central bore 93 of the collet 90. This alternative tapered embodiment has the benefit of creating a wedge effect which encourages the bone screw 80 to remain firmly attached to the collet 90 after implantation. This retention benefit can also be achieved by placing a small flange, protrusion (e.g., bumps), threads, ridges, grooves, furrows, channels or indentations around the exterior circumference at the top 84 of the bone screw shaft and/or placing a similar flange, protrusion, threads, ridges, grooves, furrows, channels or indentations around the interior circumference at the bottom 94 of the collet 90. Other gripping techniques may include creating a frictional surface by roughening or knurling the surface of the bone screw shaft 82 or collet 90. As an additional alternative, rather than fitting around the outside of the bone screw shaft 82, a central longitudinal bore (not shown) can be made inside the bone screw shaft 82 and the collet 90 can then be formed to adjustably fit inside such a central longitudinal bone screw bore.

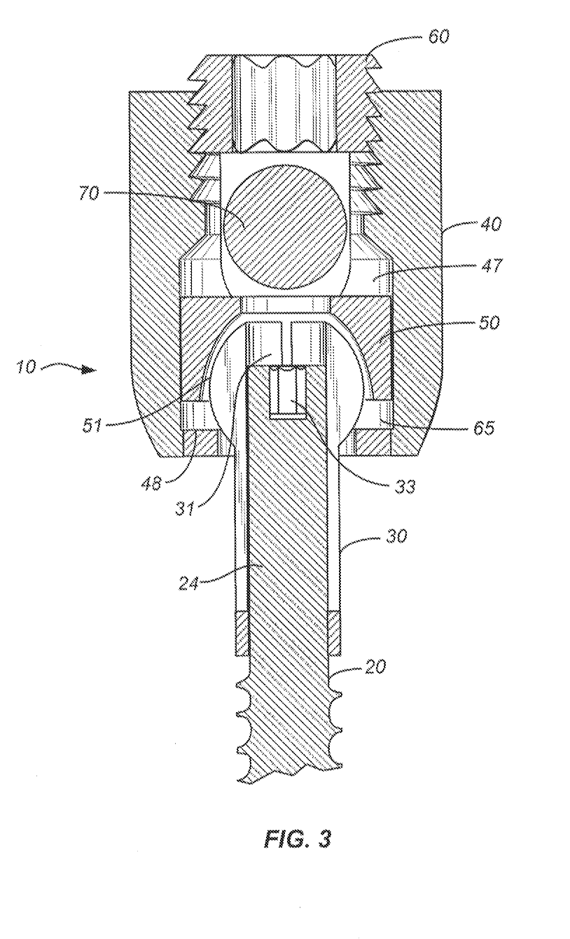

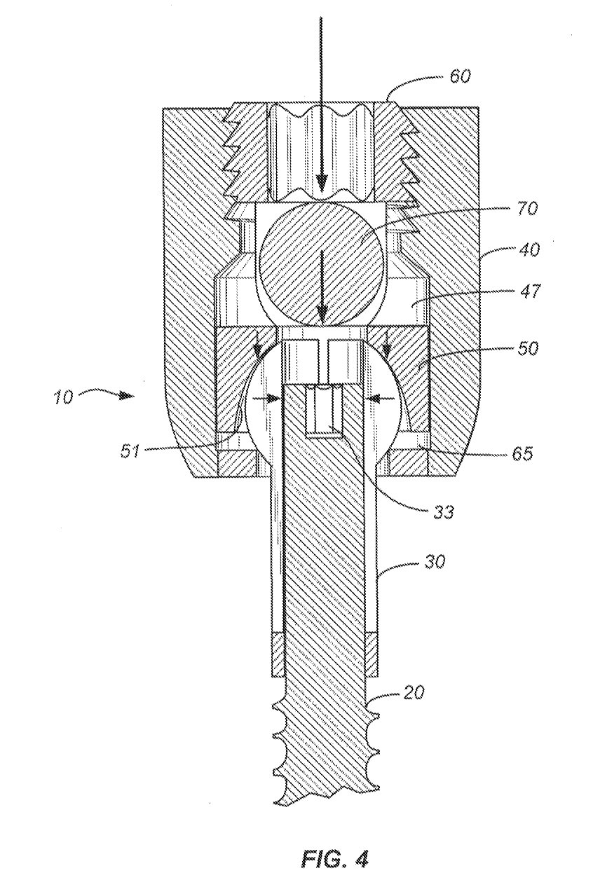

Turning now to FIGS. 3-4, a tool receiving recess 33 can be formed at the top of the bone screw shaft 24. In the embodiments shown in FIGS. 3-4, this recess 33 is a hex recess to receive a hex end driving tool. It is understood, though, that the tool receiving recess 33 can have other configurations, such as a TORX® configuration.

The distance between the threaded shank 22 of the bone screw 20 and the tulip shaped connector 40 when the bone screw assembly 10 of the present invention is fully assembled and tightened (see, FIG. 4) is determined by the collet 30. A close-up view of this collet 30, in a preferred form, is shown in FIG. 11. In this FIG. 11 embodiment, the collet 30 has a cylindrically shaped lower end 32 and a generally bulb shaped upper end 34. Inside the collet, a central bore 31 is made in a size and shape to comfortably, but snugly, fit the bone screw shaft 24. Like the bone screw 20, the collet 30 is preferably made of a strong, durable and non-infectious material, such as titanium, titanium alloy or stainless steel.

To help the collet 30 firmly grip the bone screw shaft 24 at a desired position and hold it there, a number of different slots 36 can be formed beginning at the upper end 34 of the collet and extend downward. In the preferred embodiment shown in FIG. 11, one to four such equidistant slots 36 can be used from beginning at the top of the collet bulb 34 and extending well into the cylindrically shaped lower end 32 of the collet bulb 34 (but not completely to the bottom of that lower end 32). Furthermore, these slots 36 may not be contiguous but vary in length from one another. These slots 36 create movable sections 39 of the collet 30, which are particularly movable at the bulb shaped upper end 34. When pressure is applied to the outside of these movable sections 39, they converge inwardly to grab the bone screw shaft 24.

As shown in FIG. 11, the generally bulb shaped upper end 34 of the collet is preferably flattened at the top. It has been found that making the top of the collet 30 generally bulb shaped is optimum for providing multi-axial angular variations to the position of the bone screw 20 relative to a spinal rod 70 when the bone screw assembly 10 of the present invention is fully assembled as shown in FIGS. 3-4. In one embodiment, the collet bulb 34 has a diameter of approximately 0.3 inches. As shown in FIG. 11, though, the collet bulb 34 does not form a complete sphere, but is instead preferably flattened at the top. This flattening is done to allow the collet bulb 34 to fit more securely in the tulip shaped connector 40.

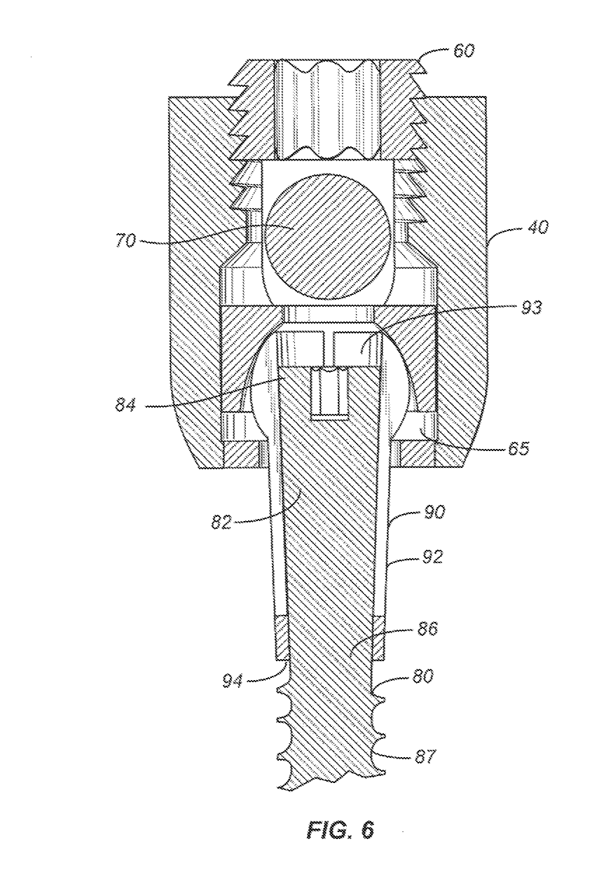

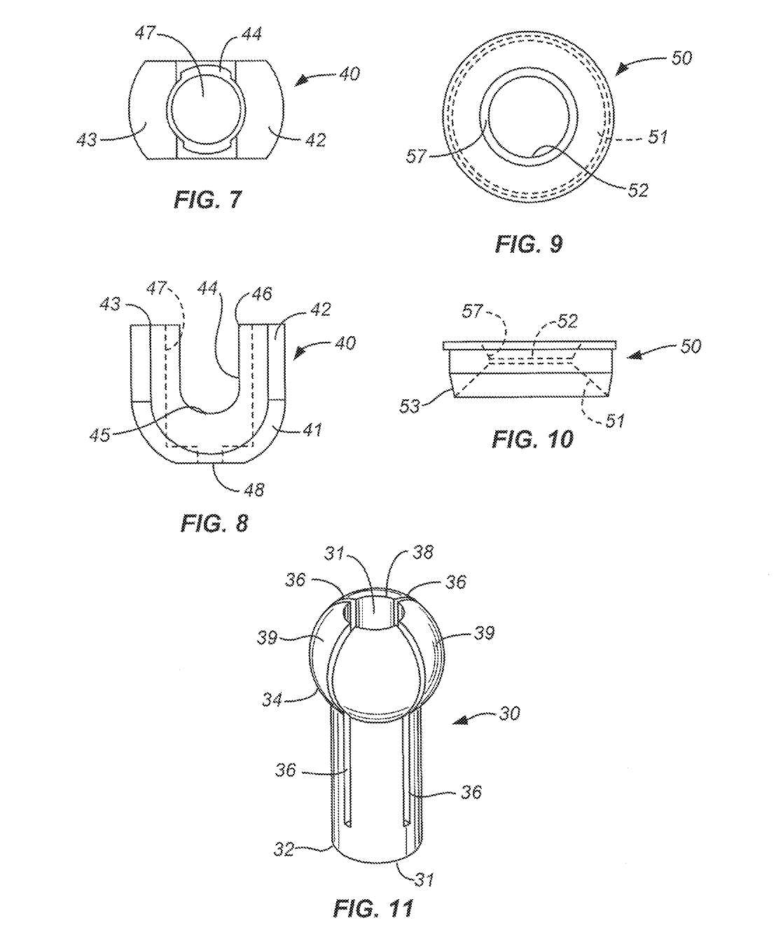

Referring again to FIGS. 2-4, a tulip shaped connector 40 is provided to support both the collet 30 and the spinal rod 70. A close-up of this tulip shaped connector 40 is provided in FIGS. 7 and 8. In the preferred embodiment shown in FIGS. 7-8, the tulip shaped connector 40 includes a U-shaped body 41 defining a first branch 42 and a second branch 43. The branches form a U-shaped channel 44 between each other. The U-shaped channel 44 terminates in a trough bottom 45. Preferably, the U-shaped channel 44 has a width that is slightly larger than the diameter of the spinal rod 70. The U-shaped channel 44 has an opening 46 at the top where rod 70, bone screw 20, collet 30 and crown member 50 can be inserted.

The tulip shaped connector 40 further defines a central bore 47. The lowermost portion of the bore 47 defines a recess having a bottom 48 within which the collet bulb 34 resides when the bone screw assembly of the present invention is fully assembled (see, FIG. 4). In addition to the collet bulb 34, the central bore 47 also accommodates the crown member 50. In one embodiment, the exterior of the crown member 50 and the crown member receiving portion of the central bore 47 may be threaded to more securely attach the crown member 50 to the tulip shaped connector 40. The tulip shaped connector 40 is preferably sized for minimal bulk and minimum prominence above the spine. In one embodiment, the tulip shaped connector 40 has a height of about 0.6 inches. In this embodiment, a rod disposed within a U-shaped channel 46 can sit as low as 0.2 inches above the surface of the vertebra when the tulip shaped connector 40 contacts the bone.

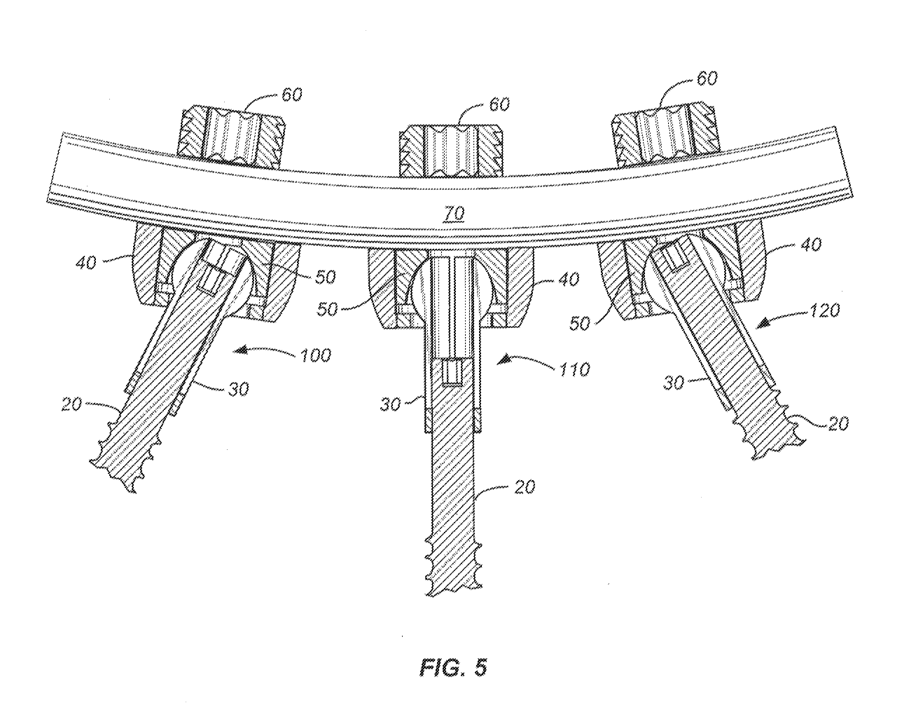

As illustrated in FIGS. 4 and 5 of Sherman's U.S. Pat. No. 5,885,286, the disclosure of which is hereby incorporated by reference, a tulip shaped connector can have tool recesses in each of its branches 42 and 43. These tool recesses are configured to be engaged by an insertion tool, such as an insertion tool used to insert spinal hooks into the spine. The tulip shaped connector 40 can also define a number of gripping holes at laterally adjacent sides of its body. These gripping holes can be engaged by an appropriately configured gripping tool to support the tulip shaped connector during tightening of the bone screw assembly 10.

As shown in FIGS. 3-4, the bone screw assembly 10 on the present invention preferably includes a crown member 50 that is positioned in the central bore 47 of the tulip shaped connector 40 between the rod 70 and the collet bulb 34. The purpose of this crown member 50 is to work in conjunction with the locking nut 65 or central bore bottom 48 to securely hold the collet bulb 34 in place and, when the set screw 60 is tightened, to exert pressure to push the sections 39 of the collect bulb 43 against the shaft 24 of the bone screw 20 to securely hold the bone screw 20 in place.

A close-up view of the crown member 50 is shown in FIGS. 9-10. In the preferred embodiment, the crown member 50 is hollow and defines a conical bore 51 at its lower end. As shown in FIGS. 3-4, the collet bulb 34 at least partially resides within the conical bore 51 of the crown member 50 when the bone screw assembly 10 is assembled. The crown member 50 further define a tool insertion bore 52 that can be oriented directly over the tool receiving recess 33 of the bone screw 20 when the bone screw 20 is situated within the tulip shaped connector 40. Crown member 50 may also defines a conical tool relief 57 at the top of the tool insertion bore 52. This relief is oriented at an angle to permit positioning of a driving tool into the tool receiving recess 33 of the bone screw 20 even when the tulip shaped connector 40 is not directly aligned with the bone screw. In another embodiment, the crown member 50 defines a spherical bore at its lower surface for contacting the collet bulb 34.

The bone screw assembly 10 of the present invention can be assembled in at least two different ways, either through the top of the tulip shaped connector 40 or partially through the bottom of the tulip shaped connector 40. Referring now to FIGS. 3-4, the top assembly method can begin by placing the locking nut 65 into the bottom 48 of the central bore recess 47. The lower end 32 of the collet can then be dropped through the hole 66 in the locking nut 65 until the bottom of the collet bulb 34 contacts the inner periphery 67 of the locking nut. The locking nut 65 should be sized so that its hole 66 is smaller than the diameter of the collet bulb 34. On the other hand, the hole 66 of the locking nut 65 should be larger than the cross-sectional diameter of the cylindrically shaped lower end 32 of the collet 30 and larger than the bottom 48 of the central bore 47 of the tulip shaped connector 40. In this way, the locking nut 65 will be held inside the tulip shaped connector 40 and also serve to hold the collet bulb 34 inside the tulip shaped connector 40, but not the lower end 32 of the collet 30. As an alternative embodiment, if the diameter of the tulip shaped connector bore bottom 48 is small enough to firmly hold the collet bulb inside the tulip shaped connector, but not the lower end 32 of the collet 30, the locking nut 65 can be dispensed with in this top assembly method.

After the locking nut 65 (if needed) and collet bulb 34 have been inserted into the central bore recess 47 of the tulip shaped connector 40, the crown member 50 is placed on top of the collet bulb 34 as shown in FIGS. 3-4. In order to prevent pieces of the tulip shaped connector 40 from falling out during assembly, the crown member 50 can have peripheral screw threads which mate with threads on the interior wall of the tulip shaped connector bore 47. By screwing the crown member 50 into the central bore 47, the locking nut 65 (if needed), collet bulb 34 and crown member 50 are prevented from falling out of the tulip shaped connector 40 while other pieces of the bone screw assembly 10 are being assembled.

Next, the rod 70 is placed in the U-shaped channel 44 of the tulip shaped connector 40 so that it rests on top of the crown member 50. A set screw 60 can then be loosely screwed into the top of the U-shaped channel 44 of the tulip-shaped connector 40. At this point, the upper shaft 24 of the bone screw 20 can be slid into the inner bore 31 of the collet 30 to a desired height and angular orientation. As previously noted, the spherical nature of the collet bulb 34 allows the collet bulb 34 to pivot to a desired orientation within the tulip shaped connector 40. When the bone screw 20 has been set to a desired height and angular orientation vis-à-vis the tulip shaped connector 40, the set screw 60 can be tightened down to lock all the components of the bone screw assembly 10 in place. As shown by the arrows in FIG. 4, the downward forces exerted by the set screw 60 are translated into angular forces by the crown member 50 to firmly press the sections 39 of the collet against the bone screw shaft 24 so that the bone screw shaft 24 is firmly held in place at its desired height and angular orientation.

The partial bottom assembly method differs in several ways from the previously described top assembly method. In the partial bottom assembly method, the collet bulb 34 is inserted through the bottom 48 of the central bore 47 into the tulip shaped connector 40. In contrast to the top assembly method, the diameter of the bottom 48 of the central bore 47 always needs to be greater than the diameter of the collet bulb 34. The locking nut 65 is next squeezed down to a smaller diameter by virtue of gap 68 and also inserted through the bottom 48 of the central bore 47. After the locking nut 65 passes through the bottom 48 of the central bore 47, it is allowed to expand back to it normal size so that it can prevent the collet bulb 34 from dropping out of the tulip shaped connector 40. The remaining steps of the partial bottom assembly method would then be the same as the top assembly method with the crown member 50 being fitted on top of the collet bulb 34 followed by the rod 70 and set screw 60. As in the top assembly method, the shaft 24 of the bone screw 20 is inserted into the collet bore 31 at a desired height and orientation before the set screw is fully tightened.

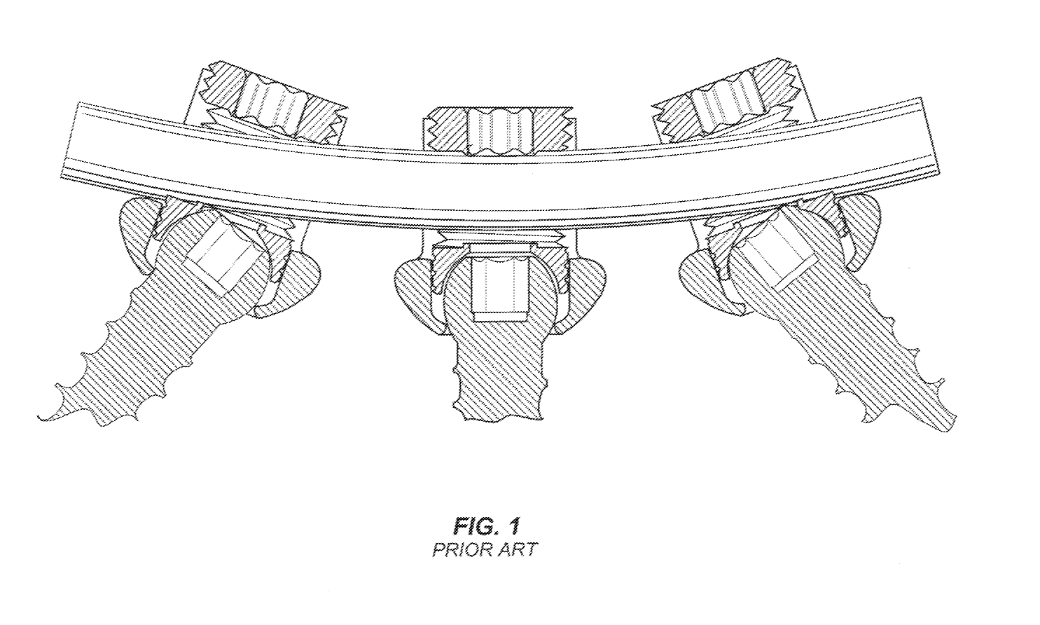

FIG. 5 illustrates how a plurality of bone screw assemblies 100, 110, 120 of the present invention can advantageously be assembled together during spinal surgery. Each of these bone screw assemblies 100, 110, 120 are connected together by a common rod 70. In the preferred embodiment, the rod 70 can either be a metal, such as titanium, titanium alloy or stainless steel, or a resilient medical plastic. By comparing the prior art bone screw assemblies shown in FIG. 1 with the bone screw assemblies of the present invention shown in FIG. 5, one can appreciate an important advantage of the present invention. As shown in FIG. 5, the interactions of the collet 30 with the bone screw 20 allows the bone screws to be fitted at different heights vis-à-vis the tulip shaped connectors 40. In particular, the left and right bone screw assemblies 100, 120 in FIG. 5 have the bone screw 20 closer to the tulip shaped connector 40 that the center bone screw assembly 110. By permitting these variable height screws, the lower surfaces of the set screws 60 and upper surfaces of the crown members 50 make flush contact with the adjacent surfaces of the rod 70. By contrast, as shown in FIG. 1, the prior art bone screw assemblies, which do not allow the bone screws to be at variable heights, can cause gaps to be formed between the adjacent surfaces of the set screw and rod as well as between the adjacent surfaces of the crown member and rod. As previously noted, these gaps can lead to a dangerous loosening of the bone screw assemblies.

In the foregoing specification, the invention has been described with reference to specific preferred embodiments and methods. It will, however, be evident to those of skill in the art that various modifications and changes may be made without departing from the broader spirit and scope of the invention. For example, while a set-screw 60 has been described for the preferred embodiment to lock the bone screw assembly 10 together, those of skill in the art will recognize that alternative types of locking compression members could also be used, such as a snap lock compression member. Also, while the bone screw 20/collet 30 combination of the present invention has been described in connection with one type of tulip shaped connector 40, those of skill in the art will readily recognize that the bone screw/collet combination of the present invention can be used with many different types of tulip shaped connectors, such as the tulip shaped connectors used by Depuy (MOUNTAINEER), Stryker (XIA), Medtronic (LEGACY, SOLARA, VERTEX) and Synthes (CLICK-X), among others. Accordingly, the specification and drawings are to be regarded in an illustrative, rather than restrictive, sense; the invention being limited only by the appended claims.

What is claimed is:

1. A bone screw assembly comprising:
a bone engaging fastener having a lower portion configured to attach to a bone and an upper portion;
a collet having a generally bulb shaped upper portion and a lower portion configured to connect to the upper portion of the bone engaging fastener at variable positions;
a connector configured to receive and retain the generally, bulb shaped upper portion of said collet while allowing angular movement of the collet along multiple axes; and,
a compression member adapted for insertion into said connector to hold said collet and said bone engaging fastener at fixed positions vis-à-vis said connector.

2. The bone screw assembly of claim 1 further comprising a rod adapted to fit into said connector and be held in fixed position by said compression member.

3. The bone screw assembly of claim 2 further comprising a crown member insertable into said connector between said rod and said generally bulb shaped upper portion of said collet.

4. The bone screw assembly of claim 3 wherein said crown member works in conjunction with said compression member to firmly hold said collet and said bone engaging fastener together.

5. The bone screw assembly of claim 1 wherein slots are formed in said collet to allow said collet to firmly hold said bone engaging fastener.

6. The bone screw assembly of claim 1 wherein said upper portion of said bone engaging fastener is tapered.

7. The bone screw assembly of claim 1 wherein said upper portion of said bone engaging fastener has a retaining flange, protrusion or indentation.

8. A method for assembling a bone screw assembly comprising the steps of:
selecting a connector with an internal bore;
inserting a collet having a generally bulb shaped upper portion and a narrower lower portion into said internal connector bore so that said generally bulb shaped upper portion is retained within said connector but said lower portion protrudes from said connector;
selecting a bone engaging fastener having a lower portion configured to attach to a bone and an upper portion configured to connect to the lower portion of said collet at variable positions;
inserting a compression member into said connector to hold said collet bulb in said connector and affix the upper portion of said bone engaging fastener to the lower portion of said collet at a desired position.

9. The bone screw assembly method of claim 8 further comprising the step of affixing a rod to said connector through use of said compression member.

10. The bone screw assembly method of claim 9 further comprising the step of inserting a crown member between said rod and said generally bulb shaped upper portion of said collet within said connector.

11. A method for assembling a variable height, multi-axial bone screw assembly:
sliding a bone screw shaft into the inner bore of a collet;
adjusting said bone screw shaft to a desired height within said collet;
inserting said collet holding said bone screw shaft into a tulip shaped connector at a desired angular orientation;
placing a rod into a U-shaped channel of said tulip shaped connector over said collet and bone screw shaft; and
screwing a set-screw tightly onto said rod to lock together all components of said bone screw assembly.
